(19) United States
(12) Patent Application Publication
(10) Pub. No.: US 2024/0016958 A1
(43) Pub. Date: Jan. 18, 2024

(54) CLOT DETECTION AND TREATMENT METHODS AND SYSTEMS

(71) Applicant: Neuravi Limited, Galway (IE)

(72) Inventors: Anushree DWIVEDI, Galway (IE); Ray MCCARTHY, Galway (IE); Sarah JOHNSON, Galway (IE)

(73) Assignee: Neuravi Limited, Galway (IE)

(21) Appl. No.: 17/865,872

(22) Filed: Jul. 15, 2022

(51) Int. Cl. A61K 49/14; A61B 5/00; A61B 6/00; A61B 34/10; A61K 49/00; G16H 20/40

(52) U.S. Cl. CPC A61K 49/14; A61B 5/0071; A61B 6/4057; A61B 34/10; A61K 49/0043; A61K 49/0056; G16H 20/40

(57) ABSTRACT

A method and system for detecting and treating a clot are disclosed. The method of detecting composition of a clot in a vessel of a patient may include introducing a solution containing tagged tissue plasminogen activator (tPA) into a circulatory system of the patient, waiting a predetermined time for the tagged tPA to interact with the clot, scanning an area of the patient corresponding to the clot, and outputting a composition of the clot based on an intensity of a signal at the clot in the scan corresponding to the tagged tPA.

### FIELD

The present invention generally relates devices, methods, for detecting and treating clots.

### BACKGROUND

There has been tremendous advancement in imaging systems which has proved beneficial for patients with Acute Ischemic Stroke (AIS). However, even with such advancement it is still difficult to precisely estimate a thrombus location, structure, and composition with currently available imaging resources. As such, there is a great need to improve visibility of a clot for a treating physician to aid the physician in deciding on an optimal treatment strategy to potentially improve treatment outcomes.

Additionally, the treatment window following the onset of a stroke for AIS patients is traditionally very short. For example, the treatment window using intravenous recombinant tissue plasminogen activator (rtPA) is up to 4.5 hours following a stroke, up to 24 hours using mechanical thrombectomy (MT) for anterior circulation strokes and for posterior strokes. There is a great need to expand the treatment window so that physician can provide timely treatment to control brain damage and improve patient outcome and recovery.

### SUMMARY

An embodiment of the present invention may include a method of detecting composition of a clot in a vessel of a patient. The method may include introducing a solution containing tagged tissue plasminogen activator (tPA) into a circulatory system of the patient followed by waiting a predetermined time for the tagged tPA to interact with the clot. RT-PA specifically targets fibrin, a main constituent of thrombi, this current invention avails of this specificity of R-tPA to localize a marker that can be used to detect an occlusive clot. The method may also include scanning an area of the patient corresponding to the clot and outputting a composition of the clot based on an intensity of a signal at the clot in the scan corresponding to the tagged tPA.

An embodiment of the present invention may include a method of detecting composition of a clot in a vessel of a patient. The method may include introducing a solution containing tagged tissue plasminogen activator (tPA) into a circulatory system of the patient, scanning an area of the patient corresponding to the clot, and outputting a composition of the clot based on an intensity of a signal at the clot in the scan corresponding to the tagged tPA.

An embodiment of the present invention may include a system for treating a clotting event. The system may include a plurality of reperfusion devices for restoring perfusion to an occluded vessel of a patient having a clot. The system may also include a delivery system for delivering at least one of a plurality of reperfusion devices to the clot in the occluded vessel. The system may further include a clot analysis system for analyzing the clot of the occluded vessel and determining an individualized treatment protocol using at least one of a plurality of reperfusion devices by scanning an area of the patient corresponding to the clot after introduction of tagged tissue plasminogen activator into a circulatory system of the patient.

tPA is an approved treatment method for AIS patients to recanalize an occluded blood vessel. Using tPA to assess composition of the clot may prove highly beneficial.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is a flow chart that schematically illustrates a method for detecting a composition of a clot in a patient.

FIGS. 2A, 2B, and 2C are diagrams of tissue plasminogen activators tagged with various biomarkers.

FIG. 3 is a diagram showing an exemplary fibrin rich clot and its corresponding imaging from a fluoroscope.

FIG. 4 is a diagram showing an exemplary red blood cell rich clot and its corresponding imaging from a fluoroscope.

FIG. 5 is a diagram showing an exemplary fibrin rich clot and its corresponding fluoroscope indicating a compact clot.

FIG. 6 is a diagram showing an exemplary red blood cell rich clot and its corresponding fluoroscope indicating clot permeability.

FIG. 7 is a diagram showing an exemplary fluoroscope that indicates a location of a clot.

### DETAILED DESCRIPTION

As used herein, the terms “component,” “module,” “system,” “server,” “processor,” “memory,” and the like are intended to include one or more computer-related units, such as but not limited to hardware, firmware, a combination of hardware and software, software, or software in execution. For example, a component may be, but is not limited to being, a process running on a processor, an object, an executable, a thread of execution, a program, and/or a computer. By way of illustration, both an application running on a computing device and the computing device can be a component. One or more components can reside within a process and/or thread of execution and a component may be localized on one computer and/or distributed between two or more computers. In addition, these components can execute from various computer readable media having various data structures stored thereon. The components may communicate by way of local and/or remote processes such as in accordance with a signal having one or more data packets, such as data from one component interacting with another component in a local system, distributed system, and/or across a network such as the Internet with other systems by way of the signal. Computer readable medium can be non-transitory. Non-transitory computer-readable media include, but are not limited to, random access memory (RAM), read-only memory (ROM), electronically erasable programmable ROM (EEPROM), flash memory or other memory technology, compact disc ROM (CD-ROM), digital versatile disks (DVD) or other optical storage, magnetic cassettes, magnetic tape, magnetic disk storage or other magnetic storage devices, or any other tangible, physical medium which can be used to store computer readable instructions and/or data.

As used herein, the term “computing system” is intended to include stand-alone machines or devices and/or a combination of machines, components, modules, systems, servers, processors, memory, detectors, user interfaces, computing device interfaces, network interfaces, hardware elements, software elements, firmware elements, and other computer-related units. By way of example, but not limitation, a computing system can include one or more of a general-purpose computer, a special-purpose computer, a processor, a portable electronic device, a portable electronic medical instrument, a stationary or semi-stationary electronic medical instrument, or other electronic data processing apparatus.

As used herein, the term “non-transitory computer-readable media” includes, but is not limited to, random access memory (RAM), read-only memory (ROM), electronically erasable programmable ROM (EEPROM), flash memory or other memory technology, compact disc ROM (CD-ROM), digital versatile disks (DVD) or other optical storage, magnetic cassettes, magnetic tape, magnetic disk storage or other magnetic storage devices, or any other tangible, physical medium which can be used to store computer readable information.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

Disclosed embodiments involve a newly developed clot fluoroscope that enables a physician to determine a clot composition, permeability and/or location prior to treatment. By using the disclosed embodiments, physicians are able to make more informed decisions when selecting a treatment strategy for their patients. For example, such information enables physicians to decide how to (i) channel the patient to the proper hospital, (ii) differentiate between ischemic and hemorrhagic strokes, (iii) estimate the location of the clot, (iv) allow accurate positioning of a clot retrieval device, (v) confirm successful clot capture, (vi) help visualize clot fragments that have embolized, and (vii) identify appropriate treatment techniques based on clot composition, permeability, and/or location.

Disclosed embodiments use fibrin-specific markers to identify fibrin content in the clot, in combination with rtPA as the carrier molecule. Because fibrin is one of the main components of a clot and rtPA has a specificity for fibrin, the rtPA with fibrin-specific markers will attach to the clot to confirm the presence of a clot, identify the composition of the clot, and location in the vasculature. The rtPA will be conjugated with a radiopaque label such as iodine, so that it is visible by imaging systems. In some embodiments an rtPA may be labeled with a metal or magnetic label which can be analyzed by an appropriate external detection system. In some embodiments, a low dose of rtPA (potentially small non-clinical dose) may be used. Plasminogen and rtPA are highly specific to fibrin surfaces. Plasminogen and rtPA can coexist in plasma but they interact in the presence of fibrin fibers. In some embodiments, the biomarker may be detected by a system present in an ambulance or a local primary care center. In some embodiments, a spectroscopic means to detect the clot may be used which may be similar to a temperature check gun that is portable and easy to carry anywhere.

rTPA may be conjugated with fluorophore flourescein isothiocyanate (FITC). Whole blood clots were treated with rtPA and the sections of clots were visible under a microscope. (See G. Jones et al., “In Vitro Investigations Into Enhancement of tPA Bioavailability in Whole Blood Clots Using Pulsed—High Intensity Focused Ultrasound Exposures,” in IEEE TRANSACTIONS ON BIOMEDICAL ENGINEERING, vol. 57, no. 1, pp. 33-36, January 2010, doi: 10.1109/TBME.2009.2028316 (incorporated by reference herein)). Biomolecules such as rtPA can be radio labelled with organophosphine fluoride acceptors using the F-labelling methodology (see Hong, H., Zhang, L., Xie, F. et al. “Rapid one-step 18F-radiolabeling of biomolecules in aqueous media by organophosphine fluoride acceptors,” NAT COMMUN 10, 989 (2019) https://doi.org/10.1038/s41467-019-08953-0 (incorporated by reference herein)) and labeling with radiorhenium (see Liu G, Hnatowich D J, “Labeling biomolecules with radiorhenium: a review of the bifunctional chelators” ANTICANCER AGENTS MED CHEM. 2007 May, 7(3):367-77. doi: 10.2174/187152007780618144. PMID: 17504162; PMCID: PMC1949414. (incorporated by reference herein)). rtPA can also be labeled with a metal, metal oxide, or metallic nucleotide. (see Marion J. Limo et al., “Interactions between Metal Oxides and Biomolecules: from Fundamental Understanding to Applications,” Chemical Reviews 2018 118 (22), 11118-11193 DOI: (incorporated by reference); Christopher D. Spicer et al., “Achieving Controlled Biomolecule—Biomaterial Conjugation,” CHEMICAL REVIEWS 2018 118 (16), 7702-7743 DOI: 10.1021/acs.chemrev.8b00253 (incorporated by reference herein)).

Fibrin rich clots can be more difficult to remove and often require multiple passes during thrombectomy. Similarly, a more compacted clot has been found to have an increased stiffness, which may in turn lead to greater difficulty in removal (see Johnson S, Chueh J, Gounis M J, et al, “Mechanical behavior of in vitro blood clots and the implications for acute ischemic stroke treatment,” JOURNAL OF NEUROINTERVENTIONAL SURGERY, Published Online First: 28 Nov. 2019 (doi: 10.1136/neurintsurg-2019-015489, incorporated by reference herein). Using the proposed markers, may show a high intensity signal under imaging for a fibrin rich clot, but a low intensity signal for a red blood cell (RBC) rich clot. Additionally, a high permeability clots may show a scattered signal and a compact clot may have low permeability and may show a buildup of signal on the proximal face of the clot. Thus, the rtPA conjugated solution may have difficulty diffusing through the clot. Put another way, the clot may not allow the rtPA conjugated solution to access the inner portion of the clot causing problems in detection of the composition of the core section of the clot. Therefore, with the help of the marker outlined in this concept, valuable information can be obtained in advance of treatment which may prove highly beneficial when making treatment decisions. It may also help to achieve improved patient outcomes and significantly reduce brain damage with the help of timely and effective treatment.

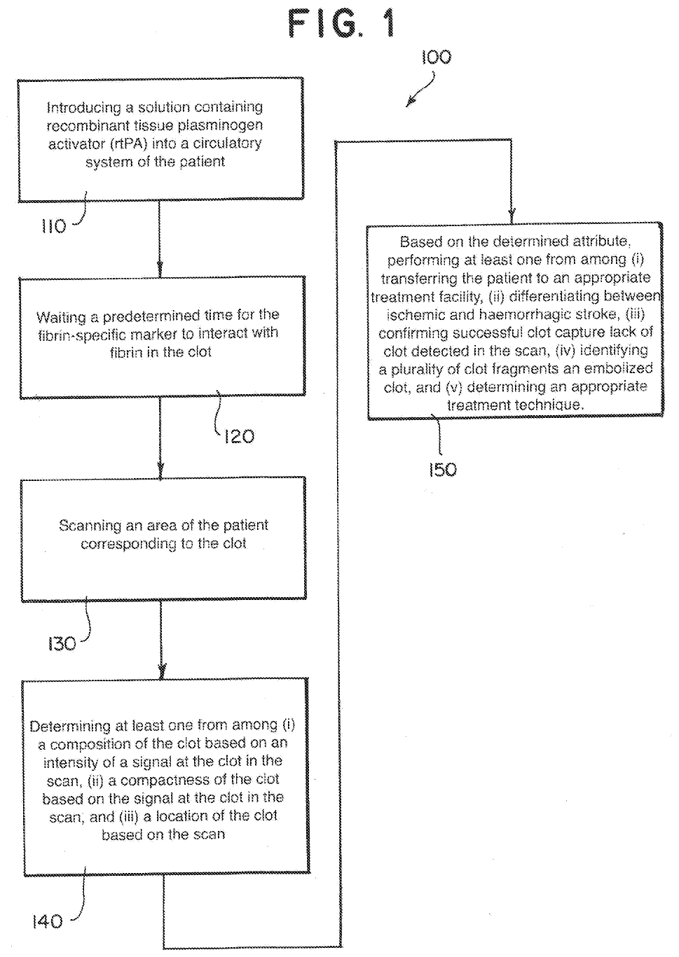

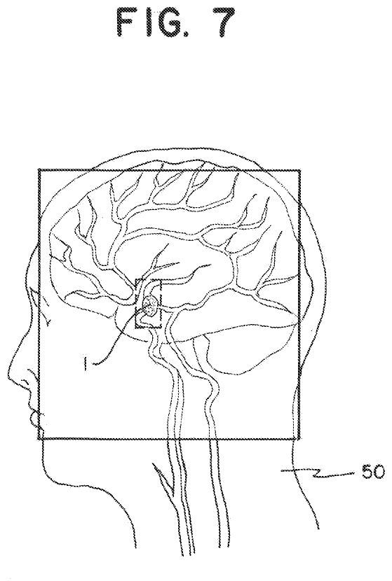

Referring FIGS. 1-7, method 100 may include introducing a solution containing a tissue plasminogen activator (tPA) 210 (e.g., a tagged recombinant tissue plasminogen activator (rtPA)) into a circulatory system of a patient 50 (step 110) and waiting a predetermined time for the tPA with a fibrin-specific marker to interact with fibrin in a patient's clot 1 in a vessel 2 (step 120). The method 100 may also include scanning an area of the patient 50 corresponding to the clot 1 (step 130) with a magnetic resonance image (MRI), real-time x-ray (e.g., fluoroscope), or computerized tomography (CT) of the area (e.g., left, right front, or back of brain brain) and determining and/or outputting at least one from among (i) a composition of the clot based on an intensity of a signal 350, 450, 550, 650 at the clot 1 in the scan which may correspond to a fibrin content of the clot 1, (ii) a compactness of the clot 1 based on the signal at the clot in the scan, and (iii) a location of the clot 1 based on the scan (step 140). Optionally the method 100 may include, based on the determined attribute, performing at least one form among (i) transferring the patient to an appropriate treatment facility, (ii) differentiating between ischemic and hemorrhagic stroke using a low dose of TPA, (iii) confirming successful clot capture by observing a lack of clot detected in the scan, and (iv) identify a plurality of clot fragments of an embolized clot, and (v) determine an appropriate treatment technique.

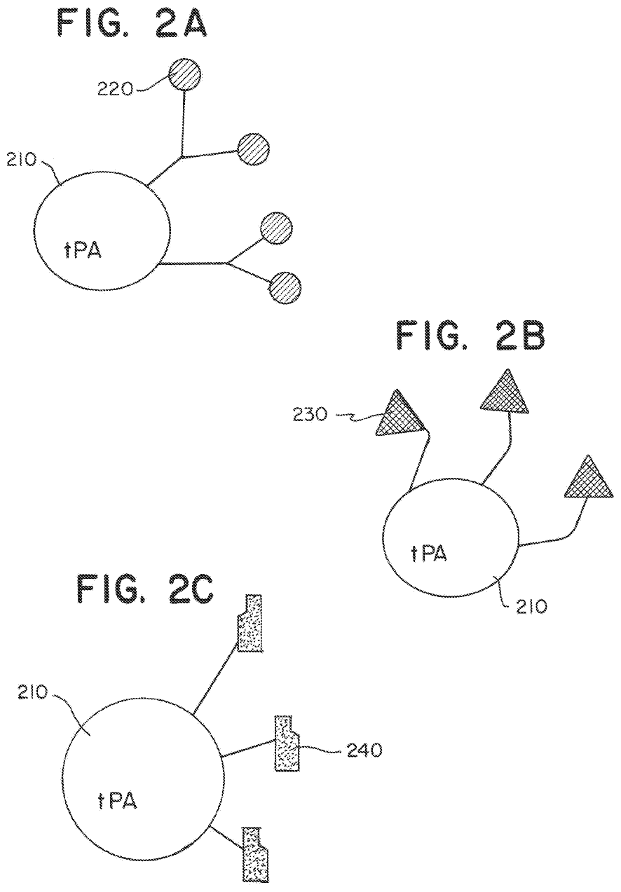

The tPA 210 may be conjugated with a biomarker 220, 230, 240 as shown in FIGS. 2A-2C. The biomarker 220, 230, 240 may be fluorophore fluorescein isothiocyanate (FITC). The tPA may be radio-labeled 230 or fluorescent labeled. The tagged tPA 210 may be labeled with an organophosphine fluoride acceptors, radiorhenium, a metal, metal oxide 240, metallic nucleotide, a magnetic label, an antibody label 220, or combinations thereof. In some embodiments the tagged tPA is labeled using an F-labelling methodology.

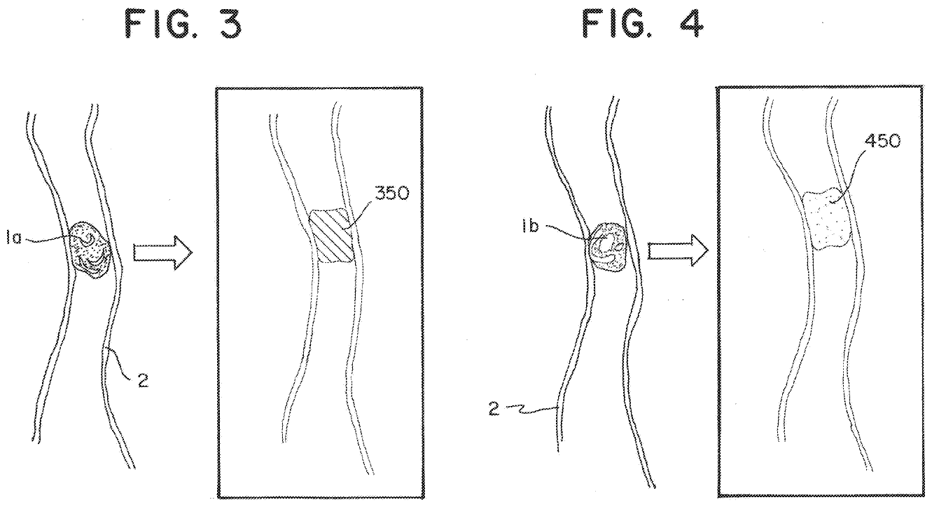

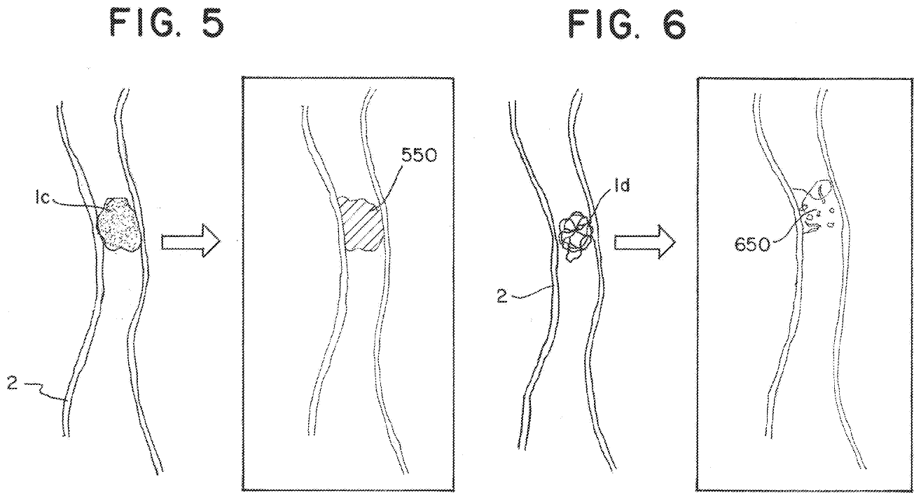

In some embodiments, a scan of the clot 1a, 1b after the tagged tPA had enough time to interact with it, may reveal a high intensity signal 350 corresponding to a fibrin rich clot or a low intensity signal 450 corresponding to an RBC rich clot, see FIGS. 3 and 4. Similarly, a compact clot 1c may correspond to a strong high intensity signal 550 and a permeable clot 1d may correspond to a scattered signal 650, see FIGS. 5 and 6.

In some embodiments, scanning the area is performed using a spectroscope. In some embodiments, scanning the area is performed using fluoroscopy.

In some embodiments, the method 100 may include identifying a location of the clot 1 based on the scan and utilizing the identified location to accurately position a clot retrieval device.

Method 100 may be performed at a primary care center and may include transferring the patient 50 to an appropriate treatment facility based on the composition of the clot 1. The method 100 may also include confirming successful clot capture in response to no clot being detected on the scan.

Method 100 may include determining individualized treatment protocol for the clot 1 based on at least one from among the composition, permeability, and location of the clot 1 and treating the clot in accordance with the individualized treatment protocol. The individualized treatment protocol may include aspirating, restoring perfusion using a first reperfusion device, restoring perfusion using a second reperfusion device, or combinations thereof. When the individualized treatment protocol includes restoring perfusion using a first reperfusion device and/or restoring perfusion using a second reperfusion device, the first reperfusion device may be a stent retriever and the second reperfusion device may be a pinch retriever. The stent retriever may be configured to remove a clot or portions of a clot that are red blood cell rich and the pinch retriever may be configured to remove a clot or portions of a clot that are fibrin-rich. In some embodiments, if the composition of the clot 1 demonstrates that the clot 1 is fibrin-rich, the individualized treatment protocol comprises passing the pinch retriever device by, through, or about the clot 1 and then retracting the pinch retriever device while pinching the clot 1 to restore reperfusion to the vessel. In some embodiments, if the composition of the clot 1 demonstrates that the clot 1 is not fibrin-rich, the individualized treatment protocol comprises passing the stent retriever by, through, or about the clot 1 and then retracting the stent retriever while engaging the clot 1 in a lumen of the stent retriever to restore reperfusion to the vessel.

In some embodiments, method 100 may further include receiving, through a graphical user interface of a computing device, the individualized treatment protocol, monitoring, by the computing device, perfusion of the vessel 2 with the clot 1; and, alerting, by the computing device, in response to perfusion being restored in the vessel 2.

In some embodiments, a system for treating a clotting event may include a plurality of reperfusion devices for restoring perfusion to an occluded vessel 2 of a patient 50 having a clot 1. The system may also include a delivery system for delivering at least one of the plurality of reperfusion devices to the clot in the occluded vessel and a clot analysis system for analyzing the clot 1 of the occluded vessel and determining an individualized treatment protocol using at least one of the plurality of reperfusion devices by scanning 130 an area of the patient 50 corresponding to the clot 1 after introduction of a tagged tissue plasminogen activator into a circulatory system of the patient.

The descriptions contained herein are examples of embodiments of the invention and are not intended in any way to limit the scope of the invention. As described herein, the invention contemplates many variations and modifications of . . . , including . . . . Modifications and variations apparent to those having skilled in the pertinent art according to the teachings of this disclosure are intended to be within the scope of the claims which follow.

What is claimed is:

1. A method of detecting composition of a clot in a vessel of a patient comprising:
introducing a solution containing tagged tissue plasminogen activator (tPA) into a circulatory system of the patient;
waiting a predetermined time for the tagged tPA to interact with the clot;
scanning an area of the patient corresponding to the clot; and
outputting a composition of the clot based on an intensity of a signal at the clot in the scan corresponding to the tagged tPA.

2. The method of claim 1, wherein:
the tPA is tagged by conjugation with a biomarker; and
the biomarker comprises fluorophore fluorescein isothiocyanate (FITC).

3. The method of claim 1, wherein the intensity of a signal corresponds to a fibrin content of the clot.

4. The method of claim 1, wherein the tagged tPA is radio-labeled or fluorescent labeled.

5. The method of claim 1, wherein the tagged tPA is labeled with at least one from among an organophosphine fluoride acceptors, radiorhenium, a metal, metal oxide, metallic nucleotide, a magnetic label, and an antibody label.

6. The method of claim 3, further comprising determining a compactness of the clot based on the signal at the clot in the scan,
wherein a strong signal corresponds to a compact clot.

7. The method of claim 1, further comprising determining a permeability of the clot based on the signal at the clot in the scan,
wherein a scattered signal corresponds to a permeable clot.

8. The method of claim 1, wherein scanning the area comprises capturing a magnetic resonance image (MRI) of the area, scanning using a spectroscope, or scanning using fluoroscopy.

9. The method of claim 1, further comprising identifying a location of the clot based on the scan.

10. The method of claim 9, further comprising utilizing the identified location to accurately position a clot retrieval device.

11. The method of claim 1, further comprising differentiating between ischemic and hemorrhagic stroke based on the scan.

12. The method of claim 1, further comprising identifying a plurality of clot fragments of an embolized clot.

13. The method of claim 1, further comprising: determining individualized treatment protocol for the clot based on at least one from among the composition, permeability, and location of the clot; and treating the clot in accordance with the individualized treatment protocol.

14. The method of claim 13, wherein the individualized treatment protocol comprises one or more techniques selected from using at least one of aspirating, restoring perfusion using a first reperfusion device, and restoring perfusion using a second reperfusion device.

15. The method of claim 14, wherein the first reperfusion device is a stent retriever configured to remove a clot or portions of a clot that are red blood cell rich and the second reperfusion device is a pinch retriever configured to remove a clot or portions of a clot that are fibrin-rich.

16. The method of claim 15, wherein, if the composition of the clot demonstrates that the clot is fibrin-rich, the individualized treatment protocol comprises passing the pinch retriever device by, through, or about the clot and then retracting the pinch retriever device while pinching the clot to restore reperfusion to the vessel.

17. The method of claim 15, wherein, if the composition of the clot demonstrates that the clot is not fibrin-rich, the individualized treatment protocol comprises passing the stent retriever by, through, or about the clot and then retracting the stent retriever while engaging the clot in a lumen of the stent retriever to restore reperfusion to the vessel.

18. The method of claim 15, further comprising:
receiving, through a graphical user interface of a computing device, the individualized treatment protocol;
monitoring, by the computing device, perfusion of the vessel with the clot; and,
alerting, by the computing device, in response to perfusion being restored in the vessel.

19. A method of detecting composition of a clot in a vessel of a patient comprising:
introducing a solution containing tagged tissue plasminogen activator (tPA) into a circulatory system of the patient;
scanning an area of the patient corresponding to the clot; and
outputting a composition of the clot based on an intensity of a signal at the clot in the scan corresponding to the tagged tPA.

20. A system for treating a clotting event, comprising:
a plurality of reperfusion devices for restoring perfusion to an occluded vessel of a patient having a clot;
a delivery system for delivering at least one of the plurality of reperfusion devices to the clot in the occluded vessel; and
a clot analysis system for analyzing the clot of the occluded vessel and determining an individualized treatment protocol using at least one of the plurality of reperfusion devices by scanning an area of the patient corresponding to the clot after introduction of a tagged tissue plasminogen activator into a circulatory system of the patient.
